(19) United States
(12) Patent Application Publication
(10) Pub. No.: US 2011/0136229 A1
(43) Pub. Date: Jun. 9, 2011

(54) Novel lectin

(75) Inventors: Nan-Wei Su, Taipei (TW); Teng-Hsu Wang, Taipei (TW)

(73) Assignee: National Taiwan University, Taipei (TW)

(21) Appl. No.: 12/803,409

(22) Filed: Jun. 26, 2010

(30) Foreign Application Priority Data
Dec. 3, 2009 (TW) 98141317

(51) Int. Cl. C12N 5/0783; C07K 14/42; C07K 1/18; B01D 15/36; G01N 33/53; C12N 5/078

(52) U.S. Cl. 435/375; 530/396; 436/501

(57) ABSTRACT

The present invention discloses a lectin, which is isolated from a plant belonging to the family Caricaceae. The lectin of the present invention includes a first subunit having a molecular weight of 38 kDa and a second subunit having a molecular weight of 40 kDa, wherein the lectin has a molecular weight in a range from 750 to 850 kDa and has a binding specificity to N-acetylgalactosamine and lactose.

### FIELD OF INVENTION

The present invention relates to a lectin, and more particularly, to a novel lectin isolated from Caricaceae.

### BACKGROUND OF THE INVENTION

Lectins were first isolated from castor beans and identified to be capable of inducing the erythrocyte agglutination. It is defined in Marrian Webster medical dictionary that a lectin is any of proteins especially of plants that are not antibodies and do not originate in an immune system but bind specifically to carbohydrate-containing receptors on cell surfaces (as of red blood cells). It is known that lectins have sugar-binding moieties for reversibly binding with sugars, and binding with specific mono- or oligo-saccharides on cell membranes or cell walls, so as to induce agglutination, mitosis or other physiological reactions. Therefore, lectins crosslink erythrocytes, and induce agglutination. Most lectins recognize multiple blood types, but some lectins induce agglutination to the specific blood type.

Various lectins are derived from different species of plants. It is currently known that Leguminosae, Rosaceae, Liliaceae, Laminaceae, Araliaceae and Apiaceae have abundant lectins. Further, there are various lectins isolated from wheat germs, corns, tomatoes, peanuts, bananas, mushrooms, rice, potatoes and etc. Lectins have specificity to bind saccharides. For example, Con A specifically binds to mannose and glucose, and the wheat germ agglutinin (WGA) specifically binds to N-acetylglucosamine. Thus, lectins are conventionally applicable to isolation or purification of glycoproteins in vivo.

Many lectins are not digested in the digestive system, and have physiological activities in the circulatory system. Lectins induce agglutination via binding to the glycoproteins on the cell membrane, so as to induce the proliferation of lymphocytes or bone marrow cells and induce the release of signal transducing factors (such as cytokines, interferons, growth factors and etc.) It is known that lectins induce the proliferation of immune cells (such as killer cells,) regulate immunoactivities (such as the secretion of cytokines, phosphorylation of proteins and etc.), affect the protein synthesis (such as binding to ribosomes for inhibiting the protein synthesis,) regulate the cell cycle, inhibit the growth of tumor cells (such as decreasing the activity of telomerase and inhibiting angiogenesis,) and induce the apoptosis of tumor cells (Gabius, Andre et al. 2002; Gabius, Siebert et al. 2004; Gonz'Alez De Mej'Ia and Prisecaru 2005; Gonz'Alez De Mej'Ia and Prisecaru 2005.) Hence, some lectins are applied for treating cancers (Bies, Lehr et al. 2004; Gabius 2004; Gabor, Bogner et al. 2004; Minko 2004; Smart 2004.)

Taiwanese Patent Application Publication No. 200538141 discloses the extract of Chaenomeles lagenaria and the preparation method thereof. The extract of Chaenomeles lagenaria includes the lectin, which specifically binds with GalNAc residue of O-linked oligosaccharides of glycophorin A. The method for preparing the extract includes the steps of using a homogenizing agent to homogenize the seeds of Chaenomeles lagenaria into a mixture, storing the mixture at 4□, centrifuging at a low speed, and filtering the supernatant. Further, Taiwanese Patent Application Publication No. 200521136 discloses a method for purifying a lectin of the marine microalgae, wherein the lectin is isolated from Chlorella luteovirides. The method includes the steps of obtaining an extract solution by cell lysis, performing precipitation by using an ammonium sulfate solution, and performing purification by using an ion exchange column and a gel filtration column.

U.S. Pat. No. 6,084,072 discloses a lectin isolated from the seed of Amaranthus caudatus. The lectin has high affinity to T antigens, and has a subunit of 36 kDa. The method for extracting the lectin includes the steps of extraction, precipitation and purification by using a DEAE-cellulose column and a Synsorb-T column. Further, U.S. Pat. No. 6,846,913 discloses lectins of 56.4 kDa and 61.8 kDa isolated from Viscumalbum coloratum, wherein the lectins have the anti-tumor activity. U.S. Pat. No. 7,045,300 discloses the lectin, MFA of 30.0 kDa, which is isolated from the bark of Maackia fauriei and specifically binds with N-acetylneuraminic acid.

### SUMMARY OF THE INVENTION

The present invention provides a lectin isolated from a plant belonging to the family Caricaceae. The lectin of the present invention includes a first subunit having a molecular weight of 38 kDa and a second subunit having a molecular weight of 40 kDa, wherein the lectin has a molecular weight in a range from 750 to 850 kDa and has a binding specificity to N-acetylgalactosamine and lactose. In one embodiment, the lectin of the present invention reversibly binds to N-acetylgalactosamine and lactose.

In one embodiment of the present invention, the lectin is isolated from the plant belonging to the family Caricaceae, wherein the plant belongs to one of the group consisting of Carica, Cylicomorpha, Jacaratia, Jarilla, Horovitziana and Vasconcellea. In one embodiment of the present invention, the lectin is isolated from the plant belong to the genus Carica. In one embodiment of the present invention, the lectin is isolated from Carica papaya.

The lectin of the present invention is isolated from the family Caricaceae. In one embodiment of the present invention, the lectin is isolated from the seeds of the plant belonging to the family Caricaceae. In one embodiment of the present invention, the lectin is isolated from the seeds of the plant belonging to the genus Carica. In one embodiment of the present invention, the lectin is isolated from the seeds of Carica papaya.

In one embodiment of the present invention, the lectin has the hemagglutination activity. In one embodiment, the lectin induces agglutination by cross-linking erythrocytes. In one embodiment of the present invention, the lectin reversibly binds to the erythrocytes.

The present invention further provides a method for isolating a lectin from a plant belonging to the family Caricaceae. The method includes the steps of preparing a crude extract of seeds of the plant, precipitating proteins from the crude extract, separating the proteins by performing an ion exchange chromatography to obtain a product, and separating the product by performing a gel filtration chromatography to obtain the lectin. In one embodiment of the present invention, the plant belongs to one selected from the group consisting of Carica, Cylicomorpha, Jacaratia, Jarilla, Horovitziana and Vasconcellea. In one embodiment of the present invention, the plant belongs to the genus Carica. In one embodiment, the plant is Carica papaya.

In one embodiment of the present invention, the step of preparing the crude extract of the seeds includes the steps of homogenizing the seeds to form a homogenized seeds, mixing the homogenized seeds with a buffer solution to form a mixture, and filtering the mixture. In one embodiment of the present invention, the buffer solution includes the phosphate buffer solution. The buffer solution includes, but is not limited to, the phosphate buffer saline. In one embodiment of the present invention, the buffer solution includes NaN₃. In one embodiment of the present invention, the seed powder is mixed with the buffer solution, and then extracted at a temperature in a range from 0 to 10□. Preferably, the mixture is extracted at 4□. In one embodiment of the present invention, the pH of the buffer solution is in a range from 5 to 9, preferably in a range from 6 to 8, and more preferably in a range from 6.8 to 7.8. In one embodiment of the present invention, the method includes the step of screening for a fraction having hemagglutination activity after preparing the crude extract.

In one embodiment of the present invention, the step of precipitating the proteins is performed by using 0 to 70% ammonium sulfate saturation, preferably by using 0 to 50% ammonium sulfate saturation, and more preferably by using 0 to 30% ammonium sulfate saturation.

In one embodiment of the present invention, the ion exchange chromatography is performed by using an ion exchange column. In one embodiment of the present invention, the ion exchange chromatography is performed by using an anion exchange column. In one embodiment of the present invention, the ion exchange chromatography is performed by using a cation exchange column. In one embodiment of the present invention, the ion exchange chromatography is performed by using a cation exchange column and an anion exchange column. In one embodiment of the present invention, the ion exchange chromatography is performed at pH 6.0 to 7.0. In one embodiment of the present invention, the ion exchange chromatography is performed with 150 to 500 mM of sodium chloride buffer solution. In one embodiment of the present invention, the buffer solution includes the phosphate buffer solution. The buffer solution can be, but not limited to, the phosphate buffer saline.

In one embodiment of the present invention, the ion exchange chromatography is performed by using an anion exchange column at pH 7.0. In one embodiment of the present invention, the ion exchange chromatography is performed by using an anion exchange column and a buffer solution including 150 mM of sodium chloride at pH 7.0. In one embodiment of the present invention, the buffer solution includes the phosphate buffer solution. The buffer solution can be, but not limited to, the phosphate buffer (such as the phosphate buffer saline.) In one embodiment of the present invention, the ion exchange chromatography is performed with 50 mM of the phosphate buffer solution. In one embodiment of the present invention, the anion exchange column includes anionic exchange resins having diethylaminoethyl groups.

In one embodiment of the present invention, the ion exchange chromatography is performed by using a cation exchange column at pH 6.0. In one embodiment of the present invention, the ion exchange chromatography is performed by using a cation exchange column and a buffer solution having 300 mM sodium chloride at pH 6.0. In one embodiment of the present invention, the buffer solution includes the phosphate buffer solution. The buffer solution can be, but not limited to, the phosphate buffer solution (such as the phosphate buffer saline). In one embodiment of the present invention, the ion exchange chromatography is performed with 50 mM of the phosphate buffer solution. In one embodiment of the present invention, the cation exchange column includes cationic exchange resins having carboxymethyl groups.

In one embodiment of the present invention, the method includes the step of screening for the product having hemagglutination activity after performing the ion exchange chromatography.

In one embodiment of the present invention, the method further includes the step of screening for the product having hemagglutination activity to obtain the lectin after performing the gel filtration chromatography. In one embodiment of the present invention, the method includes the steps of subjecting the product having hemagglutination activity screened after the gel filtration chromatography to another gel filtration chromatography, and then after the latter gel filtration chromatography, screening for a product having hemagglutination activity.

The present invention further provides a method for regulating an immune cell to secrete a cytokine. In one embodiment of the present invention, the method includes the step of interacting the immune cell with a lectin isolated from a plant belonging to the family Caricaceae. In one embodiment of the present invention, the plant belongs to one selected from the group consisting of Carica, Cylicomorpha, Jacaratia, Jarilla, Horovitziana and Vasconcellea. In one embodiment of the present invention, the plant belongs to the genus Carica. In one embodiment, the plant is Carica papaya.

In one embodiment, the lectin of the present invention includes a first subunit having a molecular weight of 38 kDa and a second subunit having a molecular weight of 40 kDa, wherein the lectin has a molecular weight of about 800 kDa and has a binding specificity to N-acetylgalactosamine and lactose. In one embodiment, the lectin of the present invention reversibly binds to N-acetylgalactosamine and lactose.

In one embodiment of the present invention, the immune cell is one selected from the group consisting of Jurkat T lymphocyte, J45.01 T lymphocyte, HuT 78 T lymphocyte, H9 T lymphocyte, CD3+ CD4+ T cell lineage, and a primary human peripheral blood mononuclear cell. In one embodiment of the present invention, the cytokine is IL-2, IL-4, IL-8, IL-10, IFN-γ, TNF-α, TNF-β and a T cell activating cellular product.

### BRIEF DESCRIPTION OF THE DRAWINGS

FIG. 1A shows the result of the anionic exchange chromatography of the crude proteins isolated from the seeds of Carica papaya according to the present invention;

FIG. 1B shows the result of the hemagglutination assay according to the present invention;

FIG. 1C shows the result of the gel filtration chromatography according to the present invention;

FIG. 1D shows the result of the SDS-PAGE according to the present invention;

FIG. 2A shows the result of the cationic exchange chromatography of the crude proteins isolated from the seeds of Carica papaya according to the present invention;

FIG. 2B shows the result of the hemagglutination assay according to the present invention;

FIG. 2C shows the result of the gel filtration chromatography according to the present invention;

FIG. 3 shows the binding specificity of the lectin to the sugars according to the present invention;

FIG. 4A shows the fractions from Superdex 200 column according to the present invention;

FIG. 4B shows HPLC chromatogram of the proteins isolated from the seeds of C. papaya according to the present invention;

FIG. 5A shows the result of the size-exclusion HPLC with Shodex Kw-804 column according to the present invention;

FIG. 5B shows the result of the SDS-PAGE of CPL according to the present invention;

FIG. 6 shows sensorgrams of the interaction between CPL and immobilized GalNAc residues by surface plasmon resonance according to the present invention; and

FIG. 7 shows the effects of CPL on the cytotoxicity and induction of cytokine production in Jurkat T lymphocytes according to the present invention.

### DETAILED DESCRIPTION OF THE PREFERRED EMBODIMENTS

The detailed description of the present invention is illustrated by the following specific examples. Persons skilled in the art can conceive the other advantages and effects of the present invention based on the disclosure contained in the specification of the present invention.

Analyses

(1) Hemagglutination (HA) Assay

The human type O erythrocytes are used for the assay. The blood of type O blood donors was subjected to the vacuum blood collection tubes containing EDTA, diluted with the phosphate buffer saline (pH 7.4, w/v being 1:5,) and then centrifuged at 600×g for 10 minutes. The supernatant was discarded. The wash step was repeated for three times. Subsequently, the erythrocytes were re-suspended with the phosphate buffer saline, counted by the blood cell counter, and adjusted to form the erythrocyte suspension with 2.0×10⁸ erythrocytes/mL.

50 μL of the sample (concentration: 100 μg/mL) was subjected to the two-fold sequential dilution, and then added to the microplate. Each well of the microplate was added with 50 μL of the erythrocyte suspension, and placed in the 37□ incubator for 30 minutes. The hemagglutination was observed.

(2) Analysis of Binding Specificity to N-acetylgalactosamine

100 μL of GalNAc-PAA (monosaccharides multivalent polymers purchased from GlycoTec) was added to the sample plate, and placed at 4□ overnight. Then, the GalNAc-PAA was attached to the bottom of the sample plate. The sample plate was washed with the phosphate buffer saline for five times to remove the unattached GalNAc-PAA. The sample plate was added with 200 μL of 1 wt % BSA, placed for 2-4 hours, and then washed with the phosphate buffer saline for five times.

100 μL of the sample to be tested was added to the sample plate, and shaking at 4□ overnight. The sample plate was then washed with the phosphate buffer saline to remove the unattached sample. The biotin-labeled GalNAc-PAA was added to the sample plate. Then, the streptavidin-horseradish peroxidase conjugate and 3,3′,5,5′-TMB were added for the color reaction, and the absorption at 450 nm was determined.

In this test, the control test was performed by using the soybean agglutinin (SBA), which specifically binds to GalNAc. The absorption of SBA (1 μg/mL) treated by the above steps was defined as 100%, and the binding specificity of the sample to N-acetylgalactosamine was accordingly determined.

(3) Analysis of Sugar-Binding Specificity

The N-acetylgalactosamine, galactose, lactose, mannose, N-acetylglucosamine solutions were respectively added to the same volume of the samples, and the final sugar concentrations of the solutions were 3.1, 6.3, 12.5, 25, 50 and 100 mM, respectively. Upon well mixing, 50 μL of the mixture of the sample and the sugar solution was added to 50 μL of the human type O erythrocyte suspension (2×10⁸ cells/mL) in the 96-well plate. Then, the 96-well plate was placed at 37□ for 30 minutes, and the inhibition of the hemagglutination was observed. IC₅₀ was defined as the lowest sugar concentration capable of inhibiting 50% agglutinating activity, wherein the inhibition area of the agglutination was at least half area of the bottom of the well.

Preparation: Isolation and Preparation of Lectins from Carica papaya

### Embodiment 1

(1) Crude Extract of Proteins

The seeds of Carica papaya were dried and powdered (homogenized) at a temperature below 50□. 50 g of the seed powder was mixed with 500 mL of the phosphate buffer solution (20 mM, pH 7.4) including 0.02% NaN₃, and shaking at 4□ overnight. Then, the mixture was centrifuged at 9,000×g for 30 minutes, and the supernatant was filtered by suction with 0.22 μm microfilter. The filtrate was collected to be the crude extract of the seeds of Carica papaya.

The crude extract was subjected to the ultrafiltration and the molecular weight fractions. Each fraction was added with the phosphate buffer saline to the original extract volume. The mixture was then subjected to the two-fold sequential dilution for the hemagglutination assay. The result of the assay showed that, in the crude extract of the seeds of Carica papaya, the portion having the hemagglutination activity was in the fraction having the molecular weight more than 50 kDa.

The ammonium sulfate powder was slowly added to each 200 mL of the fraction having the molecular weight more than 50 kDa which has the hemagglutination activity, so as to form different ammonium sulfate saturation concentrations (0-30%, 30-50%, 50-70% and 70-90%, respectively). The mixtures stood overnight, and the proteins precipitated. After centrifugation, the precipitate was collected, dissolved with a small amount of phosphate buffer saline, and then filtered with the 0.22 μm membrane. The filtrate was added with the phosphate buffer saline, and dialyzed with the 50 kDa dialysis membrane for removing the ammonium sulfate, so as to obtain the crude proteins.

The crude proteins were subjected to the hemagglutination assay. The result showed that the crude proteins precipitated by the 0-70% ammonium sulfate saturation had the hemagglutination activity, wherein the crude protein precipitated by the 0-30% ammonium sulfate saturation had the best hemagglutination activity.

The above crude proteins precipitated by the 0-70% ammonium sulfate saturation were then subjected to the subsequent purification procedure. Alternatively, the above crude proteins precipitated by the 0-70% ammonium sulfate saturation were dried and frozen for the future purification. The dried and frozen crude proteins can be dissolved in water to the original volume.

(2) Anionic Exchange Chromatography

The crude proteins precipitated by the 0-70% ammonium sulfate saturation were centrifuged at 10,000×g for removing debris. 2 mL of the crude proteins were subjected to the DEAE Sepharose Fast Flow Column (26 mm×65 mm purchased from GE Healthcare) for the ion exchange chromatography. The anionic exchange column included the weak base anionic resins having diethyl aminoethyl groups. Before performing the ion exchange chromatography, the column was balanced with 50 mM phosphate buffer saline (pH 7.0) including 15 mM NaCl for 30 minutes. Subsequently, the crude proteins were subjected to the column. The column was then eluted in sequence with 50 mM phosphate buffer saline (pH 7.0) including 150 mM NaCl and with 50 mM phosphate buffer saline (pH 7.0) including 500 mM NaCl, and the fractions were collected.

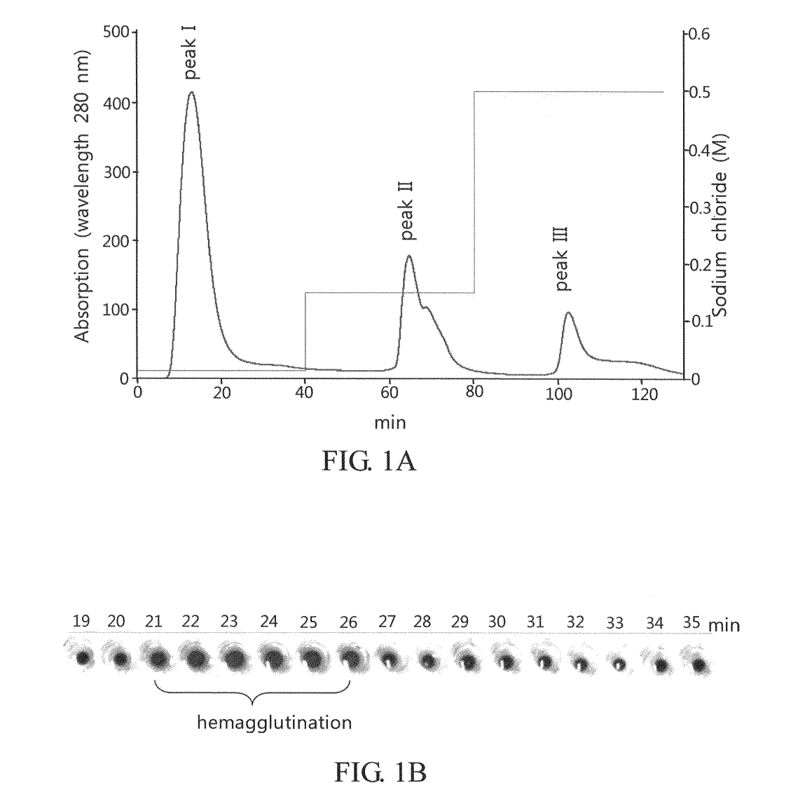

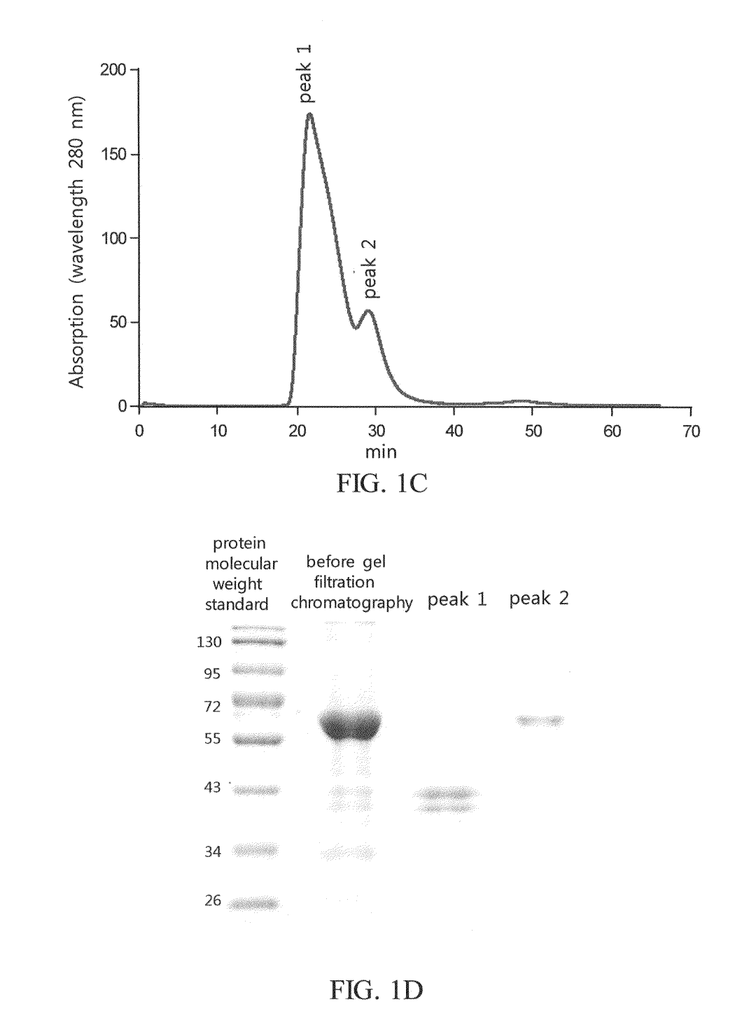

As shown in FIG. 1A, three fractions were collected. The fraction A (peak I) was eluted by the 50 mM phosphate buffer saline without sodium chloride. The fraction B (peak II) was eluted by the 50 mM phosphate buffer saline including 150 mM NaCl. The fraction C (peak III) was eluted by the 50 mM phosphate buffer saline including 500 mM NaCl. Each fraction was subjected to the hemagglutination assay. The result showed that the fraction A had the most proteins but had no hemagglutination activity; the fraction B had the hemagglutination activity; and the fraction C had no hemagglutination activity.

The above fraction B having the hemagglutination activity was subjected to the subsequent procedure.

(3) Gel Filtration Chromatography

The above fraction B was concentrated and then subjected to the column (Superdex 200 10/300 GL purchased from GE Healthcare) for the gel filtration chromatography. The mobile phase had 50 mM phosphate buffer saline (pH 7.0) including 150 mM NaCl, and the elution rate was 0.4 mL/min. The fractions were collected for the hemagglutination assay. As shown in FIG. 1B, the fractions eluted at 21-26 minutes (abbreviated as the fractions 21-26) had the significant hemagglutination activity.

The protein markers were subjected to the above mentioned gel filtration chromatography, so as to obtain the linear curve (the molecular weight of protein versus elution time.) The result showed that the fractions 21-26 had the molecular weight greater than 600 kDa. Accordingly, the product having the hemagglutination activity in the seeds of Carica papaya is the larger protein in the fraction B eluted from the anionic exchange chromatography.

The fractions 21-26 were put together, and then subjected to the above-mentioned gel filtration chromatography. As shown in FIG. 1C, the absorption peaks of the fractions were labeled as peak 1 and peak 2, wherein, as estimated above, the peak 1 indicated the molecular weight greater than 600 kDa, and the peak 2 indicated the molecular weight less than 400 kDa. The fractions of the peaks 1 and 2 were collected and concentrated for the hemagglutination assay and 15% SDS-PAGE. The result showed that the fraction of the peak 1 had the significant hemagglutination activity (HA titer being 512); and the fraction of the peak 2 had no obvious hemagglutination activity (data not shown). As shown in FIG. 1D, the result of the electrophoresis indicated that the protein having the hemagglutination activity in the seeds of Carica papaya was constituted by subunits of about 40 kDa and 38 kDa.

In light of the above results, the protein having the hemagglutination activity in the seeds of Carica papaya is constituted by subunits of about 40 kDa and 38 kDa.

### Embodiment 2

Embodiment 2 is similar to Embodiment 1 except that a cationic exchange column was used for the ion exchange chromatography.

According to the steps in Embodiment 1, the crude extract of proteins was obtained, and the proteins were precipitated. The crude proteins precipitated by 0-70% ammonium sulfate saturation were centrifuged at 10,000×g for removing the debris. 2 mL of the crude proteins were subjected to the cationic exchange column (HiTrap™ CM FF 1 mL purchased from GE Healthcare) for the ion exchange chromatography. The cationic exchange column included the weak acidic anionic resins having carboxymethyl groups. Before performing the ion exchange chromatography, the column was balanced with 50 mM phosphate buffer saline (pH 6.0) including 15 mM NaCl for 30 minutes. Subsequently, the crude proteins were subjected to the column. The column was then eluted in sequence with 50 mM phosphate buffer saline (pH 6.0) including 300 mM NaCl and with 50 mM phosphate buffer saline (pH 6.0) including 500 mM NaCl, and the fractions were collected.

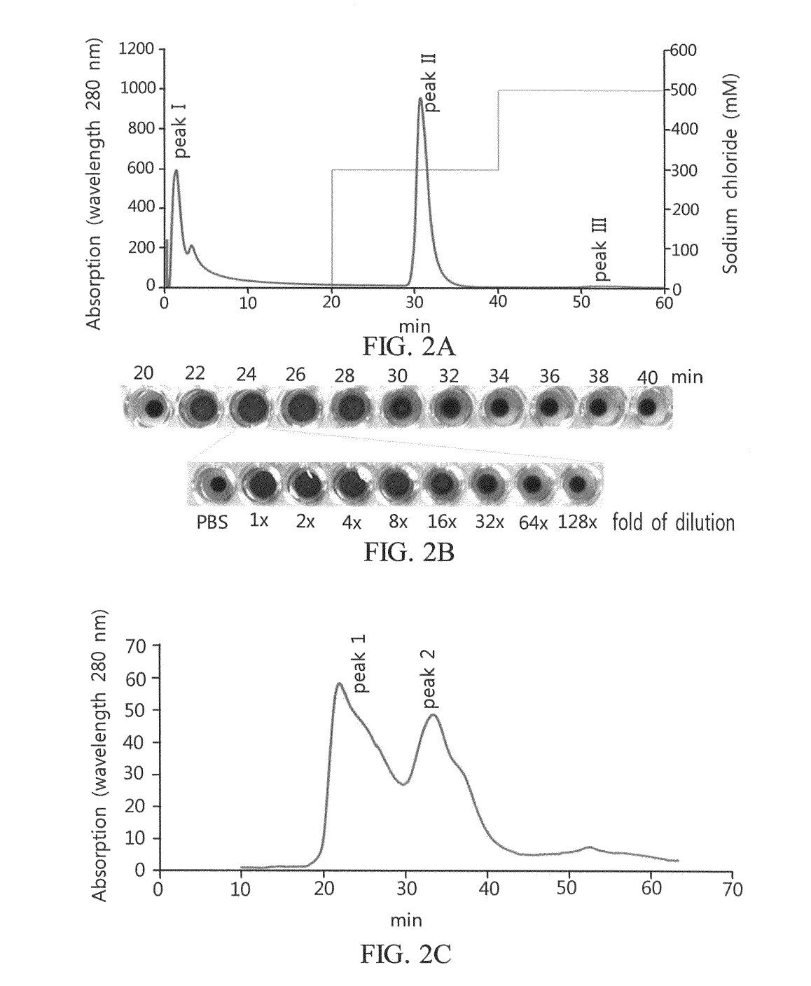

As shown in FIG. 2A, three fractions were collected. The fraction D (peak I) was eluted by the 50 mM phosphate buffer saline without sodium chloride. The fraction E (peak II) was eluted by the 50 mM phosphate buffer saline including 300 mM NaCl. The fraction F (peak III) was eluted by the 50 mM phosphate buffer saline including 500 mM NaCl. Each fraction was subjected to the hemagglutination assay. The result showed that the fraction D had the most proteins but had no hemagglutination activity; the fraction E had the hemagglutination activity; and the fraction F had no hemagglutination activity.

According to the steps in Embodiment 1, the above fraction E having the hemagglutination activity was subjected to the subsequent procedure. The fractions were collected for the hemagglutination assay. As shown in the upper portion of FIG. 2B, the fractions eluted at 21-26 minutes (abbreviated as the fractions 21-26) had the significant hemagglutination activity. The lower portion of FIG. 2B showed the result of the hemagglutination assay of the fractions having the hemagglutination activity upon different folds of dilution. Upon 16-fold dilution, the fractions still had the hemagglutination activity.

In comparison with the protein markers subjected to the gel filtration chromatography, the fractions 21-26 had the molecular weight greater than 600 kDa. Accordingly, the product having the hemagglutination activity in the seeds of Carica papaya is the larger protein in the fraction E eluted from the cationic exchange chromatography.

The fractions 21-26 were put together, and then subjected to the above-mentioned gel filtration chromatography. As shown in FIG. 2C, the absorption peaks of the fractions were labeled as peak 1 and peak 2, wherein the peak 1 indicated the molecular weight greater than 600 kDa, and the peak 2 indicated the molecular weight less than 400 kDa. The fractions of the peaks 1 and 2 were collected and concentrated for the hemagglutination assay and 15% SDS-PAGE. The result showed that the fraction of the peak 1 had the significant hemagglutination activity (HA titer being 512); and the fraction of the peak 2 had no obvious hemagglutination activity (data not shown). Further, the result of the electrophoresis indicated that the protein having the hemagglutination activity in the seeds of Carica papaya was constituted by subunits of about 40 kDa and 38 kDa (data not shown.)

The result of Embodiment 2 was consistent with that of Embodiment 1. In light of the above results, the protein having the hemagglutination activity in the seeds of Carica papaya is constituted by subunits of about 40 kDa and 38 kDa.

Moreover, in another embodiment, both the anionic exchange column of Embodiment 1 and the cationic exchange column of Embodiment 2 were used for the ion exchange chromatography. Similarly, the isolated lectins were the same as those in Embodiment 1 and Embodiment 2 (data not shown.)

Analysis of Binding Specificity

The fraction of the peak 1 in Embodiment 1 was diluted for the analysis of binding specificity to N-acetylgalactosamine and lactose. In this embodiment, the fraction of the peak 1 (HA titer being 512) was diluted for 16 folds (HA titer being 32) for the analysis.

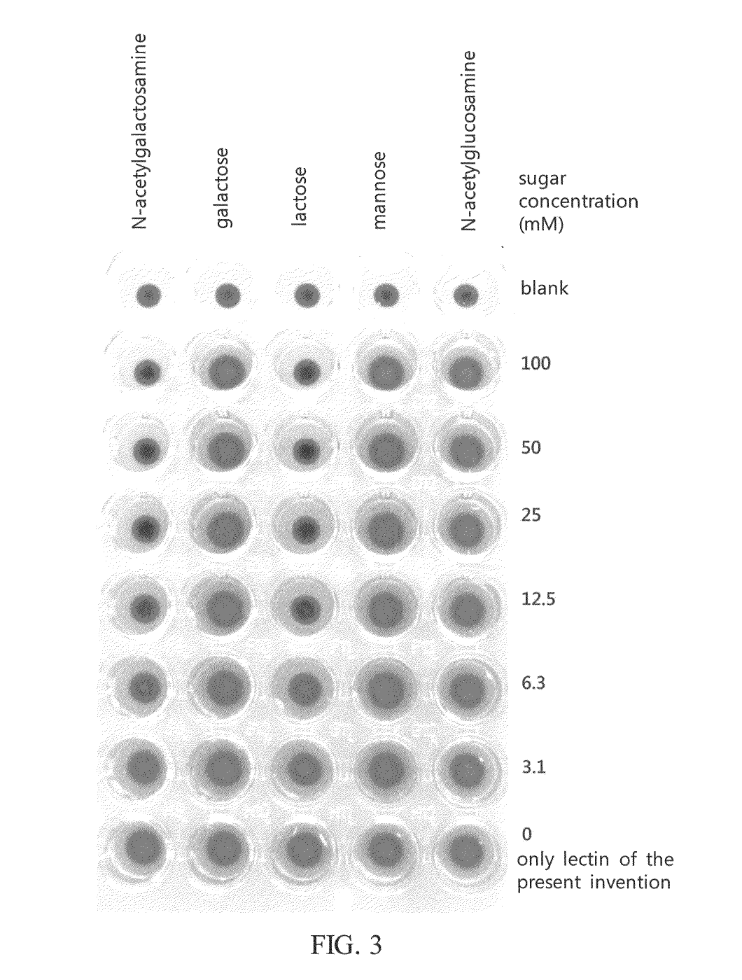

The result showed that the seeds of Carica papaya had the protein having the hemagglutination activity and the binding specificity to N-acetylgalactosamine (data not shown.) As shown in FIG. 3 and Table 1, 50% of the hemagglutination caused by the lectin of Carica papaya was inhibited by 6.3 mM (IC₅₀) of the N-acetylgalactosamine and lactose. In contrast, the IC₅₀ values of galactose, mannose and N-acetylglucosamine were more than 100 mM. The result showed that the lectin of Carica papaya had specific affinity to N-acetylgalactosamine and lactose, such that N-acetylgalactosamine and lactose competed with the sugar groups on the membrane of the erythrocyte to affect the hemagglutination activity of the lectin of Carica papaya.

TABLE 1

| Sugar | IC₅₀ (mM) |
|---|---|
| N-acetylgalactosamine | 6.3 |
| Galactose | >100 |
| Lactose | 6.3 |
| Mannose | >100 |
| N-acetylglucosamine | >100 |

Further, the fraction of the peak 1 in Embodiment 2 was diluted for the analysis of binding specificity. The similar result was obtained (data not shown.)

In addition, in one embodiment, the anionic exchange column in Embodiment 1 and the cationic exchange column in Embodiment 2 were used for the ion exchange chromatography. The fraction having the hemagglutination activity obtained from the gel filtration chromatography was diluted for the analysis of binding specificity. The similar result was obtained (data not shown.)

### Embodiment 3

(1) Purification of Lectins from Carica papaya

Embodiment 3 is similar to Embodiment 1 except that the size-exclusion HPLC is further used to determine the molecular weight of the protein having the hemagglutination activity in the seeds of Carica papaya.

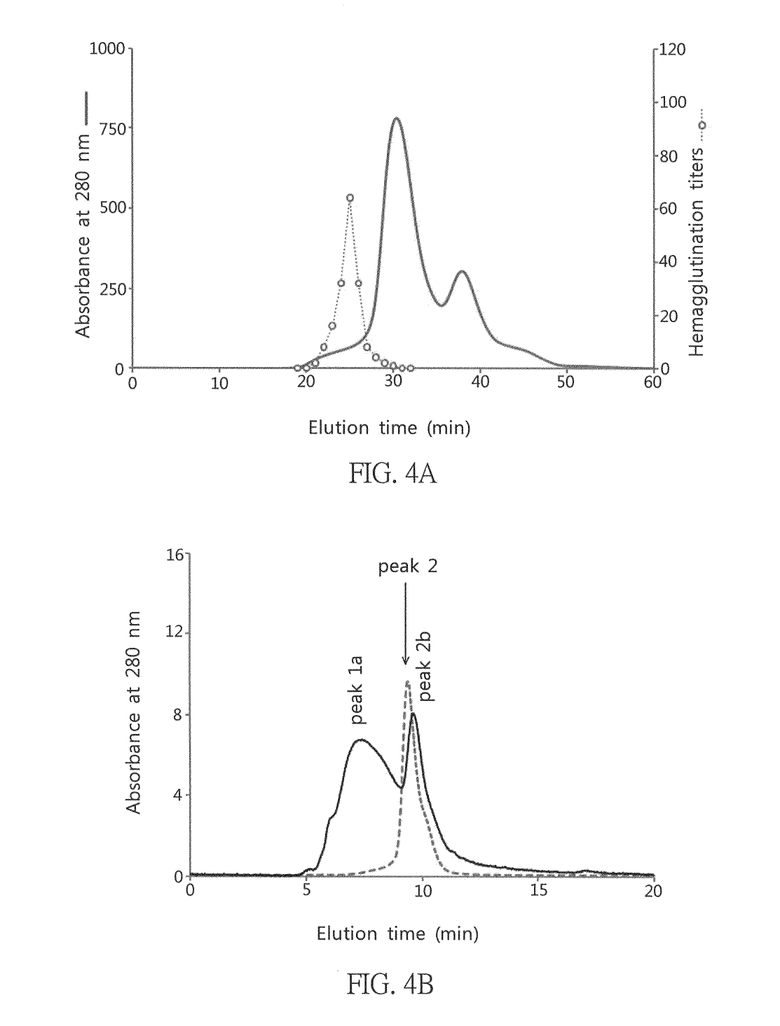

As shown in FIG. 4A, the fractions eluted at 21-26 minutes (abbreviated as the fractions 21-26) in Embodiment 1 had the significant hemagglutination activity. The protein markers were subjected to the above mentioned gel filtration chromatography in Embodiment 1, so as to obtain the linear curve (the molecular weight of protein versus elution time.) The result showed that the fractions 21-26 had the molecular weight more than 600 kDa while the elution time of blue dextran (2000 kDa) was about 20 minutes in the same conditions. Accordingly, the product having the hemagglutination activity in the seeds of Carica papaya had great molecular weight.

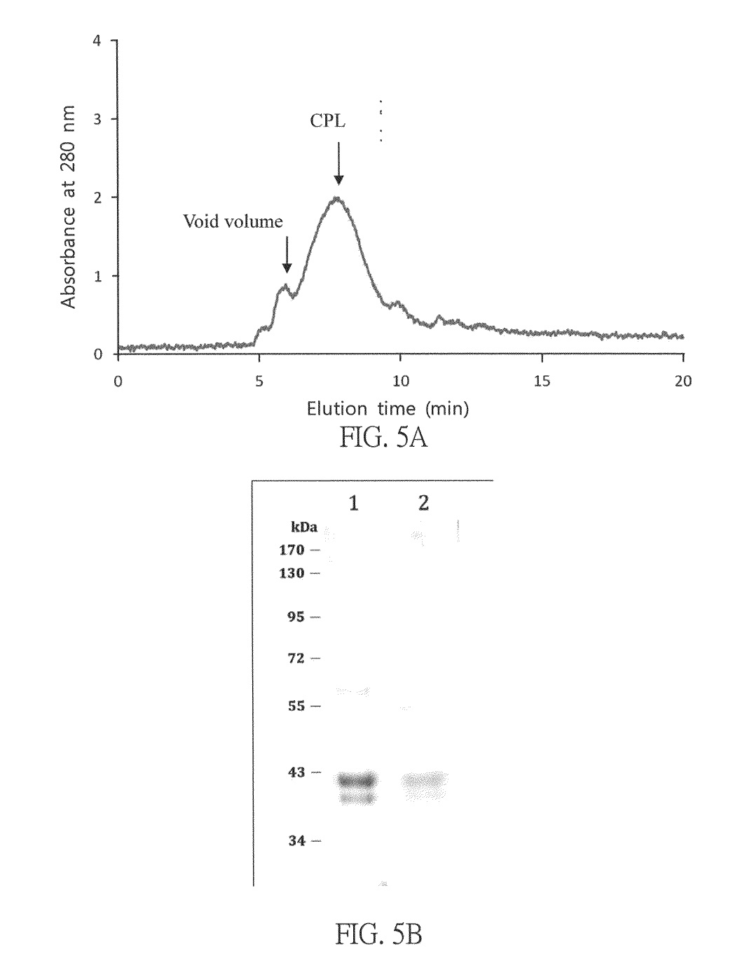

The fractions 21-26 were collected and concentrated, and then subjected to Shodex KW804 column for the size-exclusion HPLC. The results were shown in FIG. 4B, wherein the pooled active fraction indicated as a solid line having retention time of two main peaks (i.e., peak 1a and peak 1b) at about 7.5 min and about 10 min, respectively, while the removed impurities indicated as a dotted line having retention time of one main peak (i.e., peak 2) at about 10 min. Upon the hemagglutination assay, the fraction of peak 1 (i.e., the pooled active fraction of peaks 1a and 1b) showed significant hemagglutination activity, but the fraction of peak 2 had no hemagglutination activity. The fractions 21-26 were collected and concentrated, and then analyzed with 15% SDS-PAGE. The results showed that the main band of the fraction of peak 2 was at about 60 kDa, and the main bands of the fraction of peak 1 were at about 38 kDa and 40 kDa (data not shown). The fractions 21-26 were also analyzed with 6% native PAGE at pH 8.3 and pH 10.2; however, no band was shown for the fraction of peak 1 at the original molecular weight (>600 kDa). Therefore, in order to identify the molecular weight of the protein of peak 1, the fraction of peak 1 was subjected to silica-based gel filtration column Shodex Kw-804 for HPLC analysis. High Molecular Weight Gel Filtration Calibration Kit (28-4038-42, purchased from GE Healthcare) was used as the standard. In the standard proteins, bovine thyroglobulin has the largest molecular weight (669 kDa). The purified N-acetylgalactosamine binding lectin from papaya seeds had the retention time shorter than that of thyroglobulin in Kw-804 column, and was presented as a broad and symmetric peak indicated as CPL (an abbreviated name of Carica papaya lectin) in FIG. 5A. Upon calculation by extrapolation, the molecular weight of the protein of peak 1 was identified as 804±30 kDa. As shown in lane 1 of FIG. 5B, according to the result of the SDS-PAGE stained by Coomassie brilliant blue, the fraction of peak 1 was composed of about 38 kDa and 40 kDa proteins with very little impurity proteins of 60 kDa. Further, the SDS-PAGE was oxidized with periodic acid and stained by the periodic acid Schiff s reagent (Pierce Glycoprotein Staining Kit). As shown in lane 2 of FIG. 5B, the result confirmed that the proteins of about 38 kDa and 40 kDa of the present invention are glycoproteins.

Accordingly, the lectin of the present invention is glycoprotein, has the molecular weight in a range from about 774 to about 834 kDa, and is constituted by about 38 kDa and 40 kDa subunits.

The purification process of CPL is summarized in Table 2.

TABLE 2

| | | Hemaggluti- | HA | Fold of |
|---|---|---|---|---|
| | Protein | nation titers | Recovery | Purifi- |
| | (mg) | (Units/mg) | (%) | cation |
| Crude extracts of dried | 5588 | 3 | 100 | — |
| papaya seeds (200 g) | | | | |
| >50 kDa retentate | 730 | 18 | 85 | 6 |
| 0-70% Ammonium sulfate | 580 | 22 | 85 | 7.3 |
| precipitation | | | | |
| DEAE 150 mM NaCl | 72 | 89 | 43 | 29.7 |
| eluent | | | | |
| Superdex 200, the | 1 | 2560 | 17 | 853.3 |
| fractions 21-26 | | | | |

The hemagglutination titer of the crude extract of the papaya seed was about only 3 U/mg. Since the native molecular weight of CPL was estimated to be about 800 kDa, ultrafiltration (50 kDa) and 0-70% ammonium sulfate precipitation were performed to fractionate and retain proteins with relatively larger molecular weight, to increase hemagglutination titer to 22 U/mg, and to keep HA recovery rate as 85%. Upon DEAE ionic exchange chromatography, hemagglutination activity was increased to about 30 folds. After gel filtration with Superdex 200, hemagglutination activity was increased to 2560 U/mg, i.e., 850 folds.

(2) Hemagglutination Inhibition Assay on CPL by Various Sugars

The HA titer of the purified lectin in the present invention was 512. The purified lectin was diluted to have the HA titer as 16, and then mixed with various sugar solution to test hemagglutination inhibition on CPL by various sugars. The results were illustrated in Table 3, wherein MIC indicated minimum inhibitory concentrations required for inhibition of 16 hemagglutination titers of the lectin of the present invention against 2% human 0-type erythrocytes, and all sugars are of D configuration.

TABLE 3

| Sugar | MIC (mM) |
|---|---|
| GalNAc | 6.3 |
| Galactose | >100 |
| Lactose | 6.3 |
| Mannose | >100 |
| GlcNAc | >100 |

As shown in Table 3, the minimal inhibitory concentration (MIC) of sugars for inhibiting hemagglutination caused by the lectin of the invention was 6.3 mM of the N-acetylgalactosamine and lactose. In contrast, the MIC values of galactose, mannose and N-acetylglucosamine were more than 100 mM. The result showed that the lectin of the present invention had specific affinity to N-acetylgalactosamine and lactose.

(3) Interaction Between CPL and Immobilized GalNAc Residues by Surface Plasmon Resonance

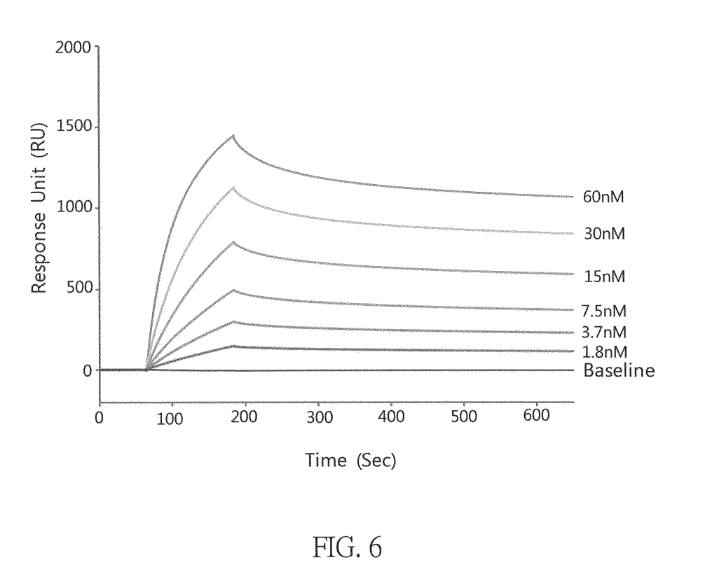

GalNAc-PAA-biotin polymers were immobilized to a sensor chip modified with streptavidin (SA). The regeneration solution for the chip was 50 mM phosphate saline buffer (pH 7.0) containing 200 mM GalNAc. The concentrations of the purified CPL were adjusted to 30, 15, 7.5, 3.7 and 1.8 nM to be analytes, then subjected to the sensor chip immobilized with GalNAc-PAA-biotin polymers, and analyzed by Biacore T100 surface plasmon resonance analyzing machine. The obtained sensorgrams were analyzed and calculated by BIACORE T100 Evaluation software 2.0. The results were shown in FIG. 6. After CPL was combined with GalNAc ligand on the sensor chip, the dissociation curve declined gradually. In other words, CPL had great affinity to GalNAc. Upon fitting calculation with BIAevaluation software, the dissociation constant (Kd) of CPL to GalNAc was about 5.5×10⁻⁹ M, the combination rate (Kₒₙ) was 5.5×10⁴ (1/MS), and the dissociation rate (K_{off}) was 3×10⁻⁴ (1/S). (4) Immunomodulatory Effect of CPL on the Cytotoxicity and Induction of Cytokine Production in Jurkat T Lymphocytes

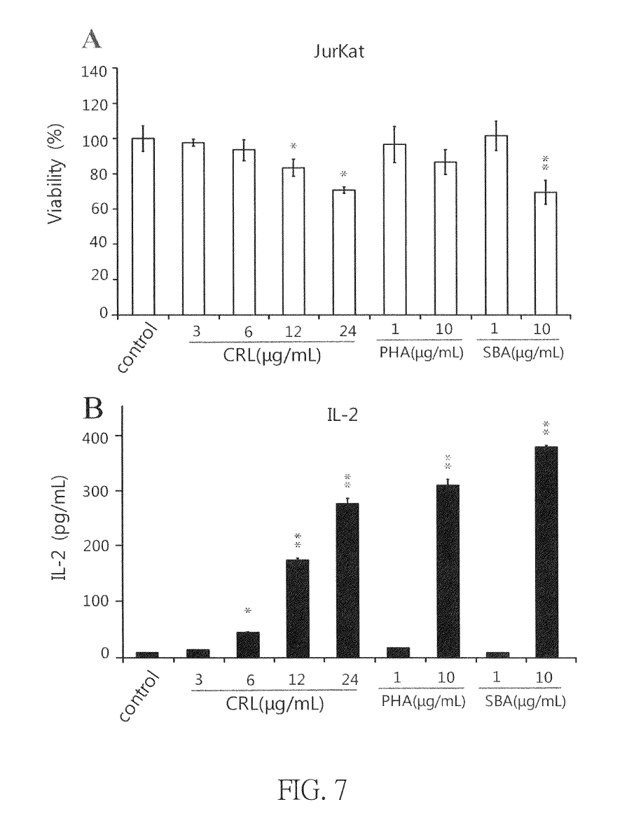

The purified CPL was added to respective 1.8 mL of Jurkat T lymphocytes (2×10⁶), wherein the final concentrations of CPL were 24, 12, 6 and 3 μg/mL, respectively. After incubation for 24 hours, cell viability was measured by cell proliferation reagent, WST-1 (4-[3-(4-iodophenyl)-2-(4-nitrophenyl)-2H-5-tetrazolio]-1,3-benzene disulfonate), and the cytokine content in the incubation medium was analyzed. The results were shown in FIG. 7. Panel A shows cell viability of Jurkat T lymphocytes. 1 μg/mL of soybean agglutinin (SBA), 1 μg/mL or 10 μg/mL of Phaseolus vulgaris phytohemagglutinin (PHA), and 3 μg/mL or 6 μg/mL of CPL had no significant effect on viability of Jurkat T lymphocytes. However, 12 μg/mL or 24 μg/mL of CPL inhibited viability of Jurkat T lymphocytes, and had significant difference from the control group (p<0.05). 24 μg/mL of CPL had inhibition effect on Jurkat T lymphocytes similar to that of 10 μg/mL of SBA, i.e., inhibiting 30% of viability after 24-hour incubation.

Panel B of FIG. 7 showed levels of IL-2 cytokine production after 24 hr treatment of lectins of the present invention in the culture medium of Jurkat T cells. In the control group of Jurkat T lymphocyte without lectin added therein, the concentration of IL-2 upon 24 hr incubation was approximately or lower than 10 pg/mL. However, upon incubation with lectin, Jurkat T lymphocytes were induced to secrete IL-2 into the culture medium. The concentrations of IL-2 secreted from Jurkat T lymphocytes incubated with SBA and PHA (10 μg/mL) were 377±1 and 308±7 pg/mL, respectively. After Jurkat T lymphocytes were incubated with various concentrations of CPL for 24 hours, the concentration of IL-2 in the medium was proportional to the concentration of CPL. In the medium added with 3 μg/mL of CPL, the concentration of IL-2 was 9.7 pg/mL, which had no significant difference from the control group. In the medium added with 6 μg/mL of CPL, the concentration of IL-2 was 44.5 pg/mL. In the medium added with 12 μg/mL and 24 μg/mL of CPL, the concentrations of IL-2 were 173±3 pg/mL and 274±9 pg/mL (p<0.001), respectively. Accordingly, CPL of the present invention induces human helper T cell line, i.e., Jurkat T lymphocytes, to secrete IL-2, and thus is capable of regulating immune cells.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

What is claimed is:

1. A lectin isolated from a plant belonging to the family Caricaceae, comprising:
a first subunit having a molecular weight of about 38 kDa; and
a second subunit having a molecular weight of about 40 kDa.

2. The lectin of claim 1, being isolated from a seed of the plant.

3. The lectin of claim 1, wherein the plant belongs to one selected from the group consisting of Carica, Cylicomorpha, Jacaratia, Jarilla, Horovitziana and Vasconcellea.

4. The lectin of claim 3, wherein the plant is Carica papaya.

5. The lectin of claim 1, having a molecular weight in a range from 750 to 850 kDa.

6. The lectin of claim 1, having a binding specificity to N-acetylgalactosamine and lactose.

7. A method for isolating a lectin from a plant belonging to the family Caricaceae, comprising the steps of:
preparing a crude extract of seeds of the plant;
precipitating proteins from the crude extract;
separating the proteins by performing an ion exchange chromatography to obtain a product; and
separating the product by performing a gel filtration chromatography to obtain the lectin.

8. The method of claim 7, wherein the step of precipitating the proteins is performed by using 0 to 70% ammonium sulfate saturation.

9. The method of claim 8, wherein the step of precipitating the proteins is performed by using 0 to 30% ammonium sulfate saturation.

10. The method of claim 7, wherein the ion exchange chromatography is performed by using one or more of a cation exchange column and an anion exchange column.

11. The method of claim 7, further comprising the step of screening for the product having hemagglutination activity.

12. The method of claim 7, wherein the plant belongs to one selected from the group consisting of Carica, Cylicomorpha, Jacaratia, Jarilla, Horovitziana and Vasconcellea.

13. The method of claim 12, wherein the plant is Carica papaya.

14. The method of claim 7, wherein the lectin has a first subunit having a molecular weight of about 38 kDa and a second subunit having a molecular weight of about 40 kDa.

15. The lectin of claim 7, wherein the lectin has a binding specificity to N-acetylgalactosamine and lactose.

16. A method for regulating an immune cell to secrete a cytokine, comprising the step of interacting the immune cell with a lectin isolated from a plant belonging to the family Caricaceae.

17. The method of claim 16, wherein the lectin has a first subunit having a molecular weight of about 38 kDa and a second subunit having a molecular weight of about 40 kDa.

18. The method of claim 16, wherein the lectin has a binding specificity to N-acetylgalactosamine and lactose.

19. The method of claim 16, wherein the immune cell is one selected from the group consisting of Jurkat T lymphocyte, J45.01 T lymphocyte, HuT 78 T lymphocyte, H9 T lymphocyte, CD3+ CD4+ T cell lineage, and a primary human peripheral blood mononuclear cell.

20. The method of claim 16, wherein the cytokine is one selected from the group consisting of IL-2, IL-4, IL-8, IL-10, IFN-γ, TNF-α, TNF-β and a T cell activating cellular product.
